(19) United States
(12) Patent Application Publication
(10) Pub. No.: US 2019/0056603 A1
(43) Pub. Date: Feb. 21, 2019

(54) TRIBOELECTRIC GENERATOR AND OPHTHALMIC LENS

(71) Applicant: HON HAI PRECISION INDUSTRY CO., LTD., New Taipei (TW)

(72) Inventor: HSIU-WEN CHIEN, New Taipei (TW)

(21) Appl. No.: 15/848,252

(22) Filed: Dec. 20, 2017

(30) Foreign Application Priority Data
Aug. 18, 2017 (CN) 201710713424.6

(51) Int. Cl. G02C 7/04; H02N 1/04

(52) U.S. Cl. CPC G02C 7/04; A61F 2/1624; H02N 1/04

(57) ABSTRACT

An ophthalmic lens producing an electrical current by means of two opposing electrode plates includes a matrix and a friction-based generator of electricity. The generator is in the matrix and includes a top electrode and a bottom electrode. The top electrode includes a polymer layer and a first metal layer stacked together. The bottom electrode includes a second metal layer facing the second metal layer. In an initial state, the second metal layer and the polymer layer are spaced apart from each other. In a contact state, the second metal layer and the polymer layer are configured to be in contact, through eye movement, to produce electricity.

### FIELD

The subject matter generally relates to a triboelectric generator and an ophthalmic lens.

### BACKGROUND

One of the major challenges with all wearable electronic devices or implanted medical devices is their frequent need to change or charge battery, which makes the wearable electronic devices or implant medical devices limited and costly. Improvement in the art is preferred.

### BRIEF DESCRIPTION OF THE DRAWINGS

Implementations of the present disclosure will now be described, by way of example only, with reference to the attached figures.

FIG. 1 is a top view of an exemplary embodiment of an ophthalmic lens according to the present disclosure.

FIG. 2 is a cross-sectional view of the ophthalmic lens of FIG. 1.

FIG. 3 shows schematic views of a triboelectric generator of the ophthalmic lens of FIG. 2.

### DETAILED DESCRIPTION OF EMBODIMENTS

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to illustrate details and features of the present disclosure better.

The disclosure is illustrated by way of example and not by way of limitation in the figures of the accompanying drawings, in which like references indicate similar elements. It should be noted that references to “an” or “one” embodiment in this disclosure are not necessarily to the same embodiment, and such references mean “at least one.”

The term “comprising,” when utilized, means “including, but not necessarily limited to”; it specifically indicates open-ended inclusion or membership in the so-described combination, group, series, and the like.

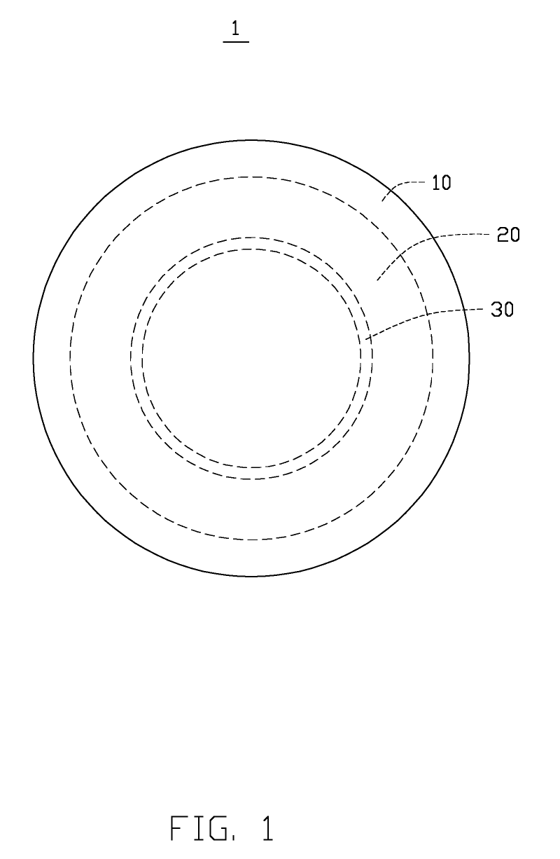

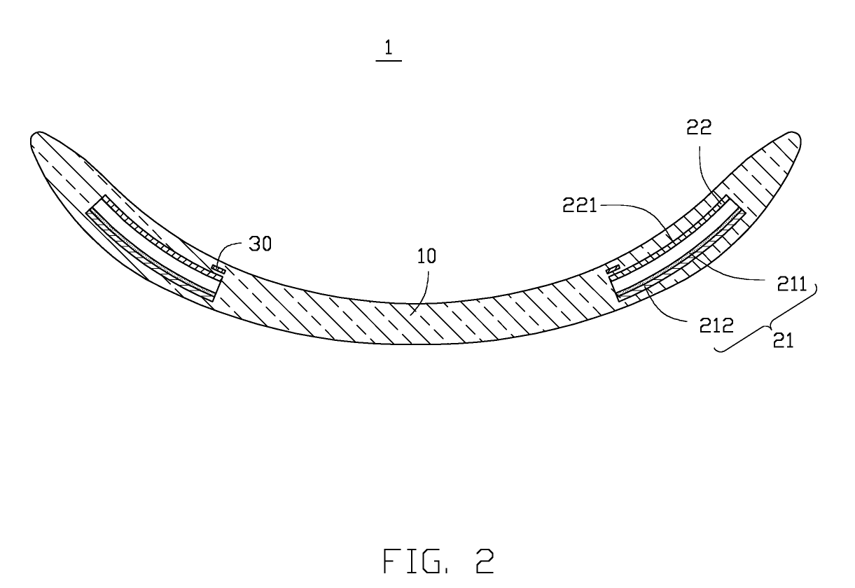

FIGS. 1 and 2 illustrate an exemplary embodiment of an ophthalmic lens 1. The ophthalmic lens 1 includes a matrix 10, a triboelectric generator 20, and a powered device 30.

The ophthalmic lens 1 may be rigid gas permeable (RGP) contact lens, soft gas permeable contact lens, ortho-K lens, or intraocular lens (IOL).

Material of the matrix 10 corresponds to the type of ophthalmic lens 1. For example, when the ophthalmic lens 1 is an RGP contact lens, the matrix 10 can be made from polymethyl methacrylate (PMMA). When the ophthalmic lens 1 is a soft gas permeable contact lens, the matrix 10 can be made from hydrogel or silicon hydrogel.

The triboelectric generator 20 and a powered device 30 are packaged into the matrix 10. The triboelectric generator 20 can supply power to the powered device 30.

The triboelectric generator 20 has a ring shape and is formed on the perimeter of the ophthalmic lens 1.

The triboelectric generator 20 includes a top electrode 21 and a bottom electrode 22 facing the top electrode 21.

In an initial state, the top electrode 21 and the bottom electrode 22 are spaced apart from each other.

The top electrode 21 includes a polymer layer 211 and a first metal layer 212. The polymer layer 211 and the first metal layer 212 are stacked together.

The polymer layer 211 is a material selected from a group consisting of polydimethylsiloxane, polytetrafluoroethylene, polymethyl methacrylate, polyethylene terephthalate, and a combination thereof.

A thickness range of the polymer layer 211 is about 1 nanometer to about 250 nanometers.

The first metal layer 212 can be aluminum, gold, or other conductive materials.

A thickness range of the first metal layer 212 is about 5 nanometers to about 45 nanometers.

The bottom electrode 22 includes a second metal layer 221. The second metal layer 221 faces the polymer layer 211. In the initial state, the second metal layer 221 and the polymer layer 211 are separated from each other.

In the initial state, a distance between the second metal layer 221 and the polymer layer 211 is about 0.4 millimeter to about 0.65 millimeter. That is, in the initial state, a distance between the top electrode 21 and the bottom electrode 22 is about 0.4 millimeter to about 0.65 millimeter.

The second metal layer 221 can be aluminum, gold, or other conductive materials.

The second metal layer 221 has a thickness range of about 5 nanometers to about 45 nanometers.

In at least one exemplary embodiment, the ophthalmic lens 1 has a diameter in a range of about 8 millimeters to about 15 millimeters.

Iris area of the ophthalmic lens 1 is defined as a central optical area of the ophthalmic lens 1 and other area which surrounds the iris area is peripheral area of the ophthalmic lens 1.

In at least one exemplary embodiment, the central optical area of the ophthalmic lens 1 has a thickness in a range of about 0.03 millimeter to about 0.7 millimeter.

In at least one exemplary embodiment, the peripheral area of the ophthalmic lens 1 has a thickness in a range of about 0.04 millimeter to about 0.85 millimeter.

In at least one exemplary embodiment, the central optical area of the ophthalmic lens 1 has a diameter in a range of about 5 millimeters to about 10 millimeters.

In at least one exemplary embodiment, the triboelectric generator 20 has a wall thickness in a range of about 2.25 millimeters to about 5 millimeters.

The powered device 30 receives the power supplied by the triboelectric generator 20.

The powered device 30 can be a sensor that detects intraocular pressure or can be a light-emitting diode screen, or other device.

When the powered device 30 is a sensor that detects intraocular pressure, the powered device 30 is formed on an area of the inner surface of the matrix 10 that is outside of the central optical area.

When the powered device 30 is a light-emitting diode screen, the powered device 30 can be set at any position of the matrix 10. For example, the powered device 30 can be packaged in the matrix 10, or formed on the inner surface of the matrix 10, or formed on the outside surface of the matrix 10.

In at least one exemplary embodiment, the ophthalmic lens 1 further includes a wire to transmit power from the triboelectric generator 20 to the powered device 30.

In other exemplary embodiment, the ophthalmic lens 1 can transmit charge generated by the triboelectric generator 20 to the powered device 30 by radio waves.

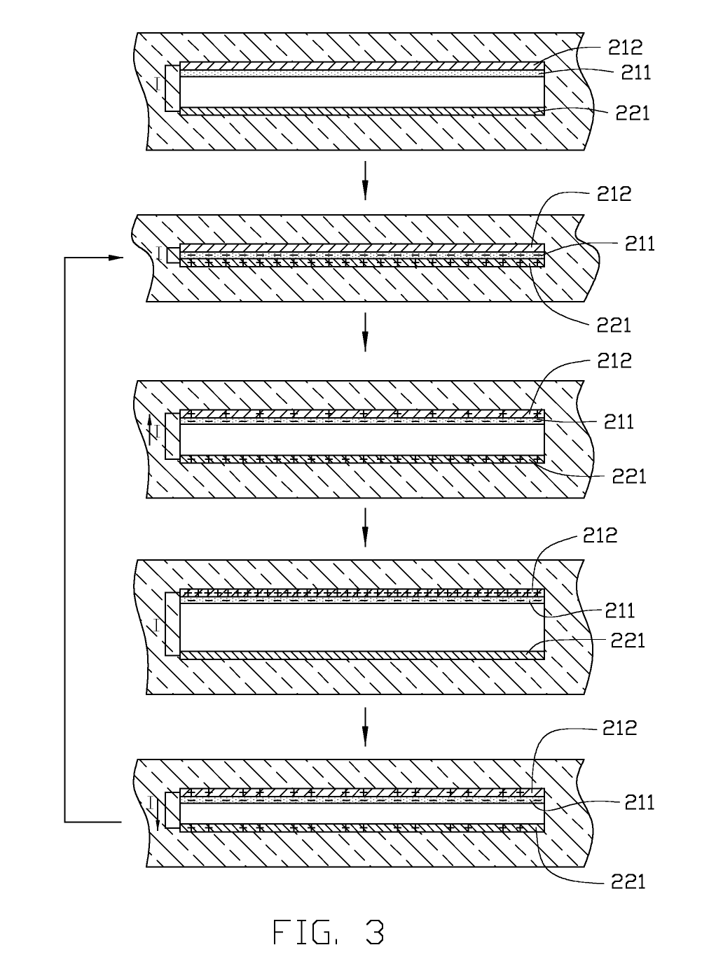

FIG. 3 illustrates a working principle of the triboelectric generator 20. When the second metal layer 221 of the bottom electrode 22 and the polymer layer 211 of the top electrode 21 are in contact with each other, a contact state, the second metal layer 221 of the bottom electrode 22 and the polymer layer 211 of the top electrode 21 induce electricity through contact. In the initial state, the second metal layer 221 of the bottom electrode 22 and the polymer layer 211 of the top electrode 21 are spaced apart from each other. Eye movement (such as blinking or eye swiveling creates an external force) brings the second metal layer 221 and the polymer layer 211 into contact, the second state. The second metal layer 221 of the bottom electrode 22 is charged positively, and the polymer layer 211 of the top electrode 21 is charged negatively by triboelectric characteristics. Removal of the external force causes the second metal layer 221 and the polymer layer 211 to separate from each other, which allows for some positive charges of the polymer layer 211 to flow to the first metal layer 212 and make the first metal layer 212 positively charged. When the second metal layer 221 and the polymer layer 211 are separated for a distance, an electrical equilibrium is formed. When the external force is applied again, which brings the second metal layer 221 and the polymer layer 211 into contact, the above process is repeated. With the repeated process, electron flow produces a current that can be transmitted to the powered device 30.

With the above configuration, the triboelectric generator 20 and the ophthalmic lens 1 can convert mechanical energy generated by blinking or rotating eyeballs into electricity, which does not require an external power supply. Moreover, according to different application fields, the electricity can be supplied to sensors, light-emitting diode screens, and other devices.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

What is claimed is:

1. A triboelectric generator, comprising:
a top electrode, the top electrode comprising a polymer layer and a first metal layer on the polymer layer;
a bottom electrode, the bottom electrode comprising a second metal layer, the second metal layer facing the polymer layer;
wherein in an initial state, the second metal layer and the polymer layer are spaced apart from each other; and
wherein in a contact state, the second metal layer and the polymer layer are configured to be in contact, through eye movement, to produce electricity.

2. The triboelectric generator of claim 1, wherein the polymer layer is a material selected from a group consisting of polydimethylsiloxane, polytetrafluoroethylene, polymethyl methacrylate, polyethylene terephthalate, and a combination thereof.

3. The triboelectric generator of claim 1, wherein the polymer layer has a thickness in a range of about 1 nanometer to about 250 nanometers.

4. The triboelectric generator of claim 1, wherein the first metal layer has a thickness in a range of about 5 nanometers to about 45 nanometers.

5. The triboelectric generator of claim 1, wherein the second metal layer has a thickness range of about 5 nanometers to about 45 nanometers.

6. The triboelectric generator of claim 1, wherein in the initial state, a distance between the second metal layer and the polymer layer is about 0.4 millimeter to about 0.65 millimeter.

7. The triboelectric generator of claim 1, wherein the triboelectric generator has a wall thickness in a range of about 2.25 millimeters to about 5 millimeters.

8. An ophthalmic lens, comprising:
a matrix;
a triboelectric generator in the matrix; wherein the triboelectric generator comprises:
a top electrode, the top electrode comprising a polymer layer and a first metal layer on the polymer layer;
a bottom electrode, the bottom electrode comprising a second metal layer, the second metal layer facing the polymer layer; wherein in an initial state, the second metal layer and the polymer layer are spaced apart from each other; and wherein in the contact state, the second metal layer and the polymer layer are configured to be in contact, through eye movement, to produce electricity.

9. The ophthalmic lens of claim 8, wherein the triboelectric generator has a ring shape and is formed on the perimeter of the ophthalmic lens.

10. The ophthalmic lens of claim 8, wherein the ophthalmic lens has a diameter in a range of about 8 millimeters to about 15 millimeters.

11. The ophthalmic lens of claim 8, wherein iris area of the ophthalmic lens is defined as a central optical area of the ophthalmic lens and other area which surrounds the iris area is peripheral area of the ophthalmic lens, the central optical area of the ophthalmic lens has a thickness in a range of about 0.03 millimeter to about 0.7 millimeter.

12. The ophthalmic lens of claim 11, wherein the peripheral area of the ophthalmic lens has a thickness in a range of about 0.04 millimeter to about 0.85 millimeter.

13. The ophthalmic lens of claim 11, wherein the central optical area of the ophthalmic lens has a diameter in a range of about 5 millimeters to about 10 millimeters.

14. The ophthalmic lens of claim 8, wherein the triboelectric generator has a wall thickness in a range of about 2.25 millimeters to about 5 millimeters.

15. The ophthalmic lens of claim 8, wherein the polymer layer is a material selected from a group consisting of polydimethylsiloxane, polytetrafluoroethylene, polymethyl methacrylate, polyethylene terephthalate, and a combination thereof.

16. The ophthalmic lens of claim 8, wherein the polymer layer has a thickness in a range of about 1 nanometer to about 250 nanometers.

17. The ophthalmic lens of claim 8, wherein the first metal layer has a thickness in a range of about 5 nanometers to about 45 nanometers.

18. The ophthalmic lens of claim 8, wherein the second metal layer has a thickness in a range of about 5 nanometers to about 45 nanometers.

19. The ophthalmic lens of claim 8, wherein in the initial state, a distance between the second metal layer and the polymer layer is about 0.4 millimeter to about 0.65 millimeter.

20. The ophthalmic lens of claim 8, wherein the ophthalmic lens further comprises a powered device in the matrix, the powered device receives power supplied by the triboelectric generator.
